(19) United States
(12) Patent Application Publication
(10) Pub. No.: US 2019/0051416 A1
(43) Pub. Date: Feb. 14, 2019

(54) Processing of Patient Health Information

(71) Applicant: Authenti-PHI, LLC, Houston, TX (US)

(72) Inventor: John Paul Monteverde, Houston, TX (US)

(73) Assignee: Authenti-PHI, LLC, Houston, TX (US)

(21) Appl. No.: 16/100,081

(22) Filed: Aug. 9, 2018

Related U.S. Application Data
(60) Provisional application No. 62/543,662, filed on Aug. 10, 2017.

(51) Int. Cl. G16H 50/30; G16H 10/60; G06Q 40/08; G06Q 20/08; G06F 17/27

(52) U.S. Cl. CPC G16H 50/30; G16H 10/60; G06F 17/2785; G06Q 20/085; G06Q 40/08

(57) ABSTRACT

Processing patient health information (“PHI”) for a patient includes receiving the PHI, scanning the PHI to produce scanned PHI, performing optical character recognition (“OCR”) and natural language processing (“NLP”) on the scanned PHI to produce structured data in order to utilize unique rules with Application Program Interfaces (“APIs”) to extract Clinical Data Elements (“CDE”) and determining from the CDE a confidence rating as to whether the patient qualifies for a Medical Product.

### CROSS-REFERENCE TO RELATED APPLICATIONS

This application claims the benefit of U.S. Provisional Application No. 62/543,662, filed Aug. 10, 2017.

### BACKGROUND

Medical records or patient health information (“PHI”) may be reviewed and/or verified in support of or in review of claims for reimbursement from insurance or governmental programs and establish medical necessity related to healthcare services, medications, devices, supplies, or products (“Medical Products”). The existing system for reviewing PHI to support a healthcare providers' or suppliers' claim for reimbursement is largely manual. For example, PHI to support medical necessity for the treatment or device and to justify payment is faxed to providers or suppliers. Once received, the PHI, sometimes consisting of hundreds of pages of medical records, is manually reviewed to locate the qualifying diagnoses, lab values, conditions, symptoms, or treatments (collectively, Clinical Data Elements or “CDE”). It is then organized and summarized for submission to insurers or governmental programs (the “payers”) to justify or authorize payment. The payers may conduct a costly and labor-intensive manual review of PHI when auditing to ensure that payment on a particular claim for a Medical Product(s) is authorized. And, for either end of the transaction, there is no reliable means of verifying that the PHI from any provider is authentic and valid. Finally, there is no efficient or cost-effective means of scanning PHI to determine the best or alternative Medical Products for which the patient's CDE may establish a medical necessity.

Reviewing and/or verifying PHI in an efficient, cost-effective, and fraud-resistant manner is a challenge.

### BRIEF DESCRIPTION OF THE DRAWINGS

The disclosure will be understood more fully from the detailed description given below and from the accompanying drawings of various embodiments, which, however, should not be taken to limit the claims to the specific embodiments, but are for explanation and understanding only.

FIG. 1 is a block diagram of a system for processing PHI.

FIG. 2 is a flow chart of a process for processing PHI.

### DETAILED DESCRIPTION

The following detailed description illustrates embodiments of the present disclosure. These embodiments are described in sufficient detail to enable a person of ordinary skill in the art to practice these embodiments without undue experimentation. It should be understood, however, that the embodiments and examples described herein are given by way of illustration only, and not by way of limitation. Various substitutions, modifications, additions, and rearrangements may be made that remain potential applications of the disclosed techniques. Therefore, the description that follows is not to be taken as limiting the scope of the appended claims. In particular, an element associated with a particular embodiment should not be limited to association with that particular embodiment but should be assumed to be capable of association with any embodiment discussed herein.

The terminology used herein is for the purpose of describing particular embodiments and is not intended to limit the scope of this application in any way. As used herein, the term “and” or “or” includes any and all combinations of one or more of the associated listed items. Furthermore, as used herein, the singular forms “a,” “an,” and “the” are intended to include the plural forms as well as the singular forms, unless the context clearly indicates otherwise. It is further understood that the terms “includes,” “including,” “compromises,” or “compromising” when used in this specification, seeks to specify the presence of stated features, steps, operations, qualities, elements, or components, but do not preclude the presence or addition of one or more other features, steps, operations, qualities, elements, or components.

Unless otherwise defined herein, all terms used have the same meaning as commonly understood by one having ordinary skill in the field to which this disclosure belongs. It is further understood that terms, such as those defined in commonly used dictionaries, should be interpreted as having a meaning that is consistent with their meaning in the context of the relevant field and the present disclosure and will not be interpreted in an idealized or overly formal sense unless expressly so defined.

A number of techniques and elements are disclosed herein, but not necessarily all elements. Each element has individual benefit and each can also be used in conjunction with one or more, or in some cases all, of the other disclosed techniques. Accordingly, for the sake of clarity, this specification will refrain from repeating every possible combination of the individual elements in an unnecessary fashion. Nevertheless, the specification and claims should be read with the understanding that such combinations are entirely within the scope of the claims.

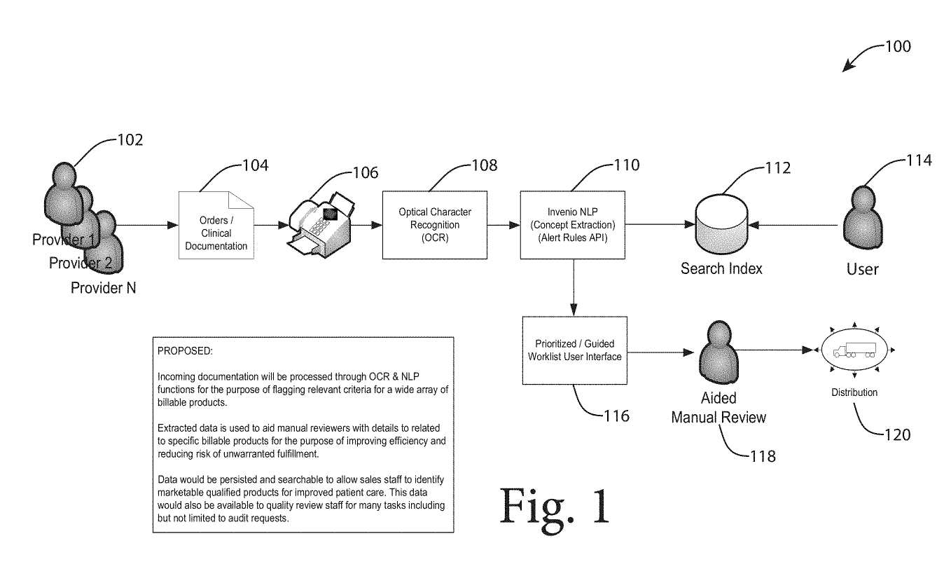

A system 100 for processing of PHI, illustrated in FIG. 1, uses optical character recognition (“OCR”) and natural language processing (“NLP”), along with coding of alert API and user interfaces to allow healthcare providers and suppliers, as well as insurers or governmental programs, to quickly review large amounts of a patient's PHI (or medical records) to extract or verify the necessary CDE that would qualify that patient for reimbursement or payment for the Medical Products ordered and provided to the patient. It also allows for an efficient and secure means of storing the PHI as well as authenticating its source.

The system 100 scans, imports, and electronically stores the patient's PHI. Providers 102 (i.e., Provider 1, Provider 2, . . . , Provider N) provide orders/clinical documentation 104 in paper form or in to some other scannable form. The documentation is scanned, imported into a storable format, and electronically stored by various devices (such as scanners) and software (such as software to convert scanning results into various formats such as text or Portable Document Format (“PDF”), Joint Photographic Experts Group (“JPEG”) or the like) 106. The system uses OCR 108 and NLP 110 to put the PHI in a searchable format (such as text known to be associated with CDE), which is then indexed in a search index 112. The search index 112 allows users 114, such as the PHI's authoring healthcare provider, the patient, or another type of user, to search and extract specific CDE from the PHI that can be used to authenticate the PHI. The same information can be used to flag relevant criteria for sales personnel to identify medically necessary and billable Medical Products that might be sold to the patient or the provider.

The system 100 allows users to set specific alert Application Programming Interfaces (“APIs”) as part of the NLP 110 based on relevant or desired Medical Product or HCPC codes for which reimbursement is sought or search other potential Medical Products for which the patient may qualify 116.

The system 100 serves two purposes: (1) to allow insurers or suppliers to verify that the qualifications for payment for any Medical Product prescribed for a patient are met based on review of the patient's medical records; and (2) provide suggestions of other potential Medical Products (cross-selling or upselling) for which the patient's medical records establish medical necessity and other necessary qualifications. The verification or recommendation process is not absolute (i.e., a simple “yes” or “no” answer) because of the variations of medical terminology and the need for analysis of both time and severity of any particular CDE (e.g.—obesity is different than morbid obesity, paralysis is different than a temporary leg injury, urinary retention can be different than a neurogenic bladder or even a notation of ‘incomplete emptying’). Instead, the system 100 provides a “confidence rating” as to whether the patient qualifies for a particular Medical Product (for example, by a percentage or red-yellow-green light symbology or the like).

The system reviews and extracts relevant patient CDE using the API prompts. In doing so, it will also give a grade or weight to each extracted term based on relevance to Medicare (or other payer) payment qualifications for a particular Medical Product or any Medical Product) as well as the number of times a particular term appears in the chart. The sum total of those word-scores from the patient's medical records (both by weight given the term and by the number of times the term appears in the patient's records) will result in a ‘confidence rating’ for each Medical Product at issue. Thus, the system 100 will use programmed medical judgment to either determine whether a patient does qualify, most likely qualifies, may qualify, or does not qualify (or additional delineating grades or ratings, as needed) for payment with his or her insurer (or suggest potential Medical Product supplies that would qualify for payment and establish medical necessity under these levels).

This use of specific, programmed, medical judgment in analyzing patient medical records improves upon the normal computing or technological process of simply extracting words or phrases performed with current NLP or OCR and is beyond any solution currently in existence and improves the way normal text extraction displays and analyzes medical data.

The system 100 provides security and fraud prevention through the use of unique user interfaces and secure storage of the PHI for each patient or specific claim. In this way, it will allow secure, electronic or web-based access to PHI by providers, suppliers, insurers/governmental programs, and patients.

The system 100 allows a user through aided manual review 118 to produce a quick reference or summary page with a bibliography of extracted information that is connected to and references the larger population of PHI which can then be distributed 120 as necessary or useful.

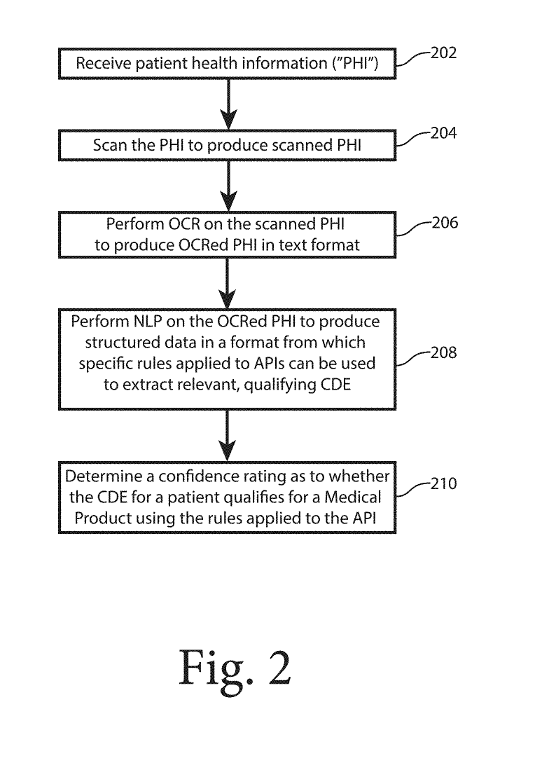

A process for processing PHI, illustrated in FIG. 2, includes receiving the PHI (block 202). The PHI is scanned to produce scanned PHI (block 204). The scanned PHI is OCRed to produce OCRed PHI in text format (block 206). NLP is then performed on the OCRed PHI to produce structured data which is data in a format from which specific rules applied to Application Program Interfaces (“API”) can be used to extract relevant, qualifying CDE (block 208). A confidence rating as to whether the CDE for a patient qualifies for a Medical Product is determined utilizing the rules applied to the API (block 210).

The confidence rating may be provided to a payer to be used in determining whether to pay on a claim, to a sales person to use to attempt to sell the Medical Product to the patient or to the payer, or to other persons as necessary or appropriate.

Performing NLP on the OCRed PHI to produce structured data from which the API and its specific rule set may analyze the PHI to identify terms of relevance to a payer or CDE, and issuing an alert indicating that such CDE was encountered. Determining the confidence rating will include associating a grade or weight to each CDE alerted based on a measure of relevance of the respective term to the payer and summing the weight and the number of times the CDE term appears in the PHI to produce the confidence rating.

The confidence rating may be in the form of a percentage, in the form of red-yellow-green light symbology, or it may specify whether the patient does qualify, most likely qualifies, may qualify, or does not qualify for payment.

The operations of the flow diagrams are described with references to the systems/apparatus shown in the block diagrams. However, it should be understood that the operations of the flow diagrams could be performed by embodiments of systems and apparatus other than those discussed with reference to the block diagrams, and embodiments discussed with reference to the systems/apparatus could perform operations different than those discussed with reference to the flow diagrams.

The text above describes one or more specific embodiments of a broader invention. The invention also is carried out in a variety of alternate embodiments and thus is not limited to those described here. The foregoing description of an embodiment of the invention has been presented for the purposes of illustration and description. It is not intended to be exhaustive or to limit the invention to the precise form disclosed. Many modifications and variations are possible in light of the above teaching.

It is intended that the scope of the invention be limited not by this detailed description, but rather by the claims appended hereto.

What is claimed is:

1. A method for processing and valuating Patient Health Information (“PHI”) for a patient in relation to payment qualifications and medical necessity guidelines, comprising:
(a) receiving the PHI;
(b) scanning the PHI to produce scanned PHI;
(c) performing optical character recognition (“OCR”) on the scanned PHI to produce OCRed PHI;
(d) performing natural language processing (“NLP”) on the OCRed PHI to produce structured data which is data that may be analyzed by Application Program Interfaces (“APIs”) to extract qualifying Clinical Data Elements (“CDE”), defined to be patient diagnoses, conditions, symptoms, treatments or other relevant medical data; and
(e) determining from scoring or grading of the specific CDE extracted a confidence rating as to whether the patient's CDE qualifies for a Medical Product, defined to be particular medical services, medications, devices, products, or supplies.

2. The method of claim 1 wherein element (e) includes providing the confidence rating for a Medical Product as it relates to the qualifications for medical necessity and payment by Medicare or other payers.

3. The method of claim 1 wherein element (e) includes providing the confidence rating to a sales person to use to attempt to sell the prescribed or an alternative or additional Medical Product to the patient, the patient's provider, or to the payer.

4. The method of claim 1 wherein performing NLP on the OCRed PHI produces structured data which the APIs then process to:
(a) identifying in the OCRed PHI a term or CDE of relevance to a payer, and
(b) scoring or grading the extracted CDE terms to produce a confidence rating for a particular Medical Product.

5. The method of claim 4 wherein determining the confidence rating includes:
(a) associating a weight to each CDE based on a measure of relevance of the respective term to the payer for establishing medical necessity and authorization for payment, and
(b) summing the weight and the number of times the CDE term appears in the PHI to produce the confidence rating as to likelihood of approval for payment by Medicare or any payer.

6. The method of claim 1 wherein the confidence rating is in the form of a percentage.

7. The method of claim 1 wherein the confidence rating is in the form of a red-yellow-green light symbology.

8. The method of claim 1 wherein the confidence rating specifies one of a plurality of levels of qualification as to whether the patient qualifies for payment.

9. A non-transitory computer-readable medium on which is recorded a computer program, the computer program comprising executable instructions, that, when executed, perform a method for processing patient health information (“PHI”) for a patient, the method comprising:
(a) receiving the PHI;
(b) scanning the PHI to produce scanned PHI;
(c) performing optical character recognition (“OCR”) on the scanned PHI to produce OCRed PHI;
(d) performing natural language processing (“NLP”) on the OCRed PHI to produce structured data which is data that may be analyzed by Application Program Interfaces (“APIs”) to extract qualifying Clinical Data Elements (“CDE”), defined to be patient diagnoses, conditions, symptoms, treatments or other relevant medical data; and
(e) determining from scoring or grading of the specific CDE extracted a confidence rating as to whether the patient's CDE qualifies for a Medical Product, defined to be particular medical services, medications, devices, products, or supplies.

10. The non-transitory computer-readable medium of claim 9 wherein element (e) includes providing the confidence rating to a payer as it relates to the qualifications for medical necessity and payment by Medicare or other payers.

11. The non-transitory computer-readable medium of claim 9 wherein element (e) includes providing the confidence rating to a sales person to use to attempt to sell the prescribed or any alternative or additional Medical Product to the patient, the patient's provider, or to the payer.

12. The non-transitory computer-readable medium of claim 9 wherein performing NLP on the OCRed PHI to produce APIs includes:
(a) identifying in the OCRed PHI a term or CDE of relevance to a payer, and
(b) scoring or grading the extracted CDE terms to produce a confidence rating for a particular Medical Product.

13. The non-transitory computer-readable medium of claim 12 wherein determining the confidence rating includes:
(a) associating a weight to each CDE term based on a measure of relevance of the respective term to the payer for establishing medical necessity and authorization for payment, and
(b) summing the weight and the number of times each specific CDE term appears in the PHI to produce the confidence rating as to likelihood of approval for payment by Medicare or any payer.

14. The non-transitory computer-readable medium of claim 9 wherein the confidence rating is in the form of a percentage.

15. The non-transitory computer-readable medium of claim 9 wherein the confidence rating is in the form of a red-yellow-green light symbology.

16. The non-transitory computer-readable medium of claim 9 wherein the confidence rating specifies one of a plurality of levels of qualification as to whether the patient qualifies for payment.
